(19) United States
(12) Patent Application Publication
(10) Pub. No.: US 2012/0259351 A1
(43) Pub. Date: Oct. 11, 2012

(54) Method of and an apparatus for restoring blood flow through a blood vessel

(76) Inventors: Mark Chak, Brooklyn, NY (US); Alexander Chak, Brooklyn, NY (US)

(21) Appl. No.: 13/066,046

(22) Filed: Apr. 6, 2011

(51) Int. Cl. A61B 17/22; A61F 2/01

(52) U.S. Cl. 606/159; 606/200

(57) ABSTRACT

Restoring a blood flow through a blood vessel includes introducing into blood flow an element which has an air and blood impermeable wall, and locating the element in the blood vessel so as to isolate a foreign substance accumulated in a blood vessel to provide an unobstructed blood flow through an interior of the element.

### BACKGROUND OF THE INVENTION

The present invention relates to a method of and an apparatus for restoring blood flow through a blood vessel.

Methods and apparatus of this type are known in the art. In known methods and apparatuses stents are inserted into a blood vessel, in which a foreign substance, such as cholesterol is deposited so that the blood vessel is expanded and a sufficient flow cross-section for blood flow is restored.

It is believed that the existing methods and apparatus can be further improved.

### SUMMARY OF THE INVENTION

Accordingly, it is an object of the present invention to provide a method of and an apparatus for restoring blood flow through a blood vessel, which are further improvements of the existing methods and apparatuses.

In keeping with these objects and with others which will become apparent hereinafter, one feature of the present invention resides, briefly stated, in a method of restoring a blood flow through a blood vessel, comprising the steps of introducing into a blood vessel an element which has an air and blood impermeable wall; and locating said element in the blood vessel so as to isolate a foreign substance accumulated in a blood vessel to provide an unobstructed blood flow through an interior of said element.

Another feature of the present invention resides in an apparatus for restoring a blood flow through a blood vessel, comprising the steps of introducing into a blood vessel an element which has an air and blood impermeable wall; and locating said element in the blood vessel so as to isolate a foreign substance accumulated in a blood vessel to provide an unobstructed blood flow through an interior of said element.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view illustrating a method in accordance with a first embodiment of the present invention;

FIG. 2 is a view illustrating a method in accordance with a second embodiment of the present invention;

FIG. 3 is a view illustrating a method in accordance with a third embodiment of the present invention;

FIG. 4 is a view illustrating a method in accordance with a fourth embodiment of the present invention;

FIG. 5 is a view illustrating a method in accordance with a fifth embodiment of the present invention; and

FIG. 6 is a view illustrating a method in accordance with a sixth embodiment of the present invention.

### DESCRIPTION OF THE PREFERRED EMBODIMENTS

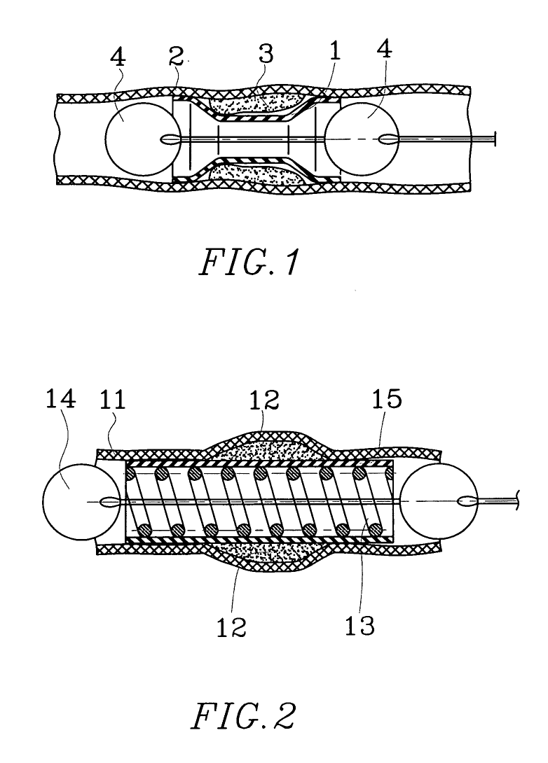

FIG. 1 shows a first embodiment of the present invention. In this embodiment an element 1 formed as a tubular, curved element is introduced into a blood vessel 2 in which a foreign deposit, for example of cholesterol 3 is formed. One expandable balloon 4 is moved forwardly of the element 1 to expand the blood vessel and to allow the insertion of the tubular element 1, and another expandable balloon 4 moves behind 1 to maintain the blood vessel expanded. The balloons are operated individually to inflate and deflate.

The tubular element 1 is composed of a material which is impermeable for air and blood. In view of this, no air or blood supply is delivered to the foreign deposit 3, and also particles of the foreign deposit 3 cannot migrate into the interior of the blood vessel thus obstructing the latter.

In the embodiment shown in FIG. 2, a blood vessel 11 has a foreign deposit such as cholesterol 12 deposited on its inner wall. An insert 13 with throughgoing openings, such as for example a spiral stent, is introduced into the blood vessel 11, being led by an inflatable balloon 14 for expanding the vessel downstream. The insert 13 and its opening are covered by a covering material 15 which is impermeable for air and blood and also for material of the foreign deposit. Therefore after the insertion 13 covered by the material 15 the previously formed deposits are reliably isolated from the interior of the blood vessel allowing a blood flow through the blood vessel. They cannot be entrained into the blood flow and also they cannot obtain a supply of air and blood to grow.

The covering material 15 can be formed as a coating or as a tubular pipe of a biocompatible, for example polymeric material attached to the insert 13 by gluing, laser welding etc.

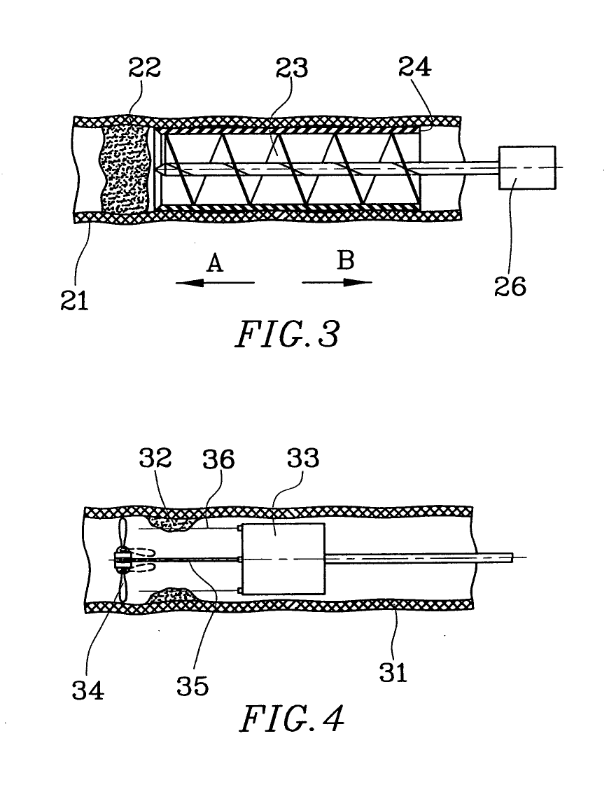

In the embodiment shown in FIG. 3 with a blood vessel 21 containing a deposit 22, a worm insert 23 penetrates into the deposit 22. A cutting tool has a body 24. The worm 23 is rotated by an electric motor 26. During advance of the device in direction A the foreign deposit is destroyed and during withdrawal of the device in direction B the material of the destroyed deposit is withdrawn through the body 24.

In an embodiment shown in FIG. 4 with the blood vessel 31 containing a deposit 32, a laser tool 33 is utilized for removing a material of the deposit 32. At its forward end the laser tool 33 has an expandable gate member 34. When the laser tool 33 is introduced into the vessel 31, the gate member 34 expands, its segments of which it is composed are turned out by action of an actuating wire 35 to form a barrier downstream of the laser tool 33. Then the laser tool destroys the deposit by a laser beam 36, and the device is pulled out using the gate member 34 as a scoop to move the material of the deposit in the withdrawal direction out of the blood vessel.

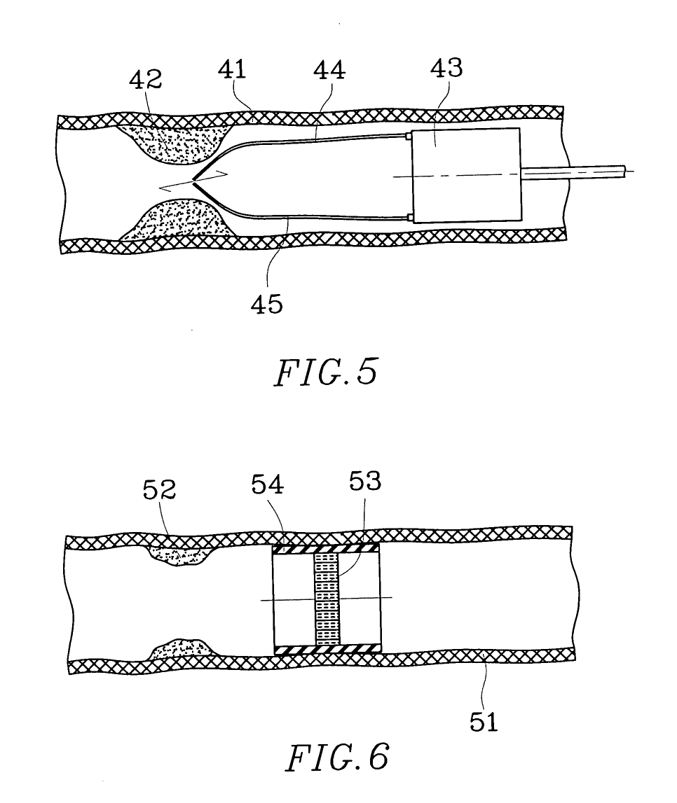

In the embodiment shown in FIG. 5 with a blood vessel 41 and foreign deposits 42, the element for restoring a blood flow in the blood vessel is formed as a device 43 which can be an electrical device provided with electrical contacts 44 and 45. During the operation the electrical contacts 44 and 45 are brought into contacts with the foreign deposit 42 and closed so as to form a closed circuit and to carry out an electrically generated heating of the deposit. This heating destroys the foreign deposit and thereby restores a blood flow through the blood vessel 41.

In the embodiment of FIG. 6 with the blood vessel 51 having a foreign deposit 52, the element is formed as a filter 53 incorporated in a tube 54. The filter can be composed of any filtering material having sufficiently small pores which prevent penetration of a foreign deposit material from one side of the filter to another side of the filter thus eliminating a possibility of propagation of the material through the blood vessel to unwanted areas. The filter 53 is provided with sensors formed for example by LEDs, which are designed so that when the filter 53 operates efficiently and allows passage of blood while filtering out the foreign deposit material, one of the LED emits one light, for example a green light, when the filter is at least partially clogged another LED is activated and emits yellow light, and when the filter is completely clogged, the third LED is activated and emits red light. These signals provide a sufficient guidance for keeping the filter and withdrawing it at proper times.

The filer 53 can have an electronic lock 54 which can be open by a coded signal sent by an authorized person, such as a doctor, to prevent its unauthorized withdrawal from the blood vessel.

It will be understood that each of the elements described above, or two or more together, may also find a useful application in other types of methods and constructions differing from the type described above.

While the invention has been illustrated and described as embodied in a method and apparatus for restoring blood flow through a blood vessel, it is not intended to be limited to the details shown, since various modifications and structural changes may be made without departing in any way from the spirit of the present invention.

Without further analysis, the foregoing will so fully reveal the gist of the present invention that others can, be applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A method of restoring a blood flow through a blood vessel, comprising the steps of introducing into blood flow a single one piece tubular element which has a single air and blood impermeable wall with an inner surface limiting an interior of said tubular one piece element and an outer surface facing a wall of the blood vessel; and locating said element in the blood vessel so as to isolate a foreign substance accumulated in a blood vessel to provide an un obstructed blood flow through an interior of said element.

1-17. (canceled)

18. A method of restoring a blood flow through a blood vessel, comprising the steps of introducing into blood flow a single one piece tubular element which has a single air and blood impermeable wall with an inner surface limiting an interior of said tubular one piece element and an outer surface facing a wall of the blood vessel; locating said element in the blood vessel so as to isolate a foreign substance accumulated in a blood vessel to provide an unobstructed blood flow through the interior of said element; providing expandable balloons at both ends of said one piece tubular element which expandable balloons are separate members which are not connected with said one piece tubular element so that when said one piece tubular element is moved in the blood vessel one of said balloons is moved forwardly of said one piece tubular element without being connected to the latter.

19. A method of restoring a blood flow through a blood vessel, comprising the steps of introducing into blood flow a single one piece tubular element which has a single air and blood impermeable wall and is composed as a whole of a single material, with an inner surface of said tubular one piece single material element limiting an interior of said tubular one piece single material element and an outer surface of said tubular one piece single material element facing a wall of the blood vessel; and locating said tubular one piece single material element in the blood vessel so as to isolate a foreign substance accumulated in a blood vessel to provide an unobstructed blood flow through an interior of said element.
